(19) United States
(12) Patent Application Publication
(10) Pub. No.: US 2011/0313255 A1
(43) Pub. Date: Dec. 22, 2011

(54) VERESS NEEDLE WITH REMOVABLE OPTICAL INSERTS

(76) Inventors: Eric Stanley, Milford, CT (US); Michael R. Davis, Middletown, CT (US); Dennis Leiner, Cape Elizabeth, ME (US); Michael Bettuchi, Middletown, CT (US); Russell Heinrich, Madison, CT (US)

(21) Appl. No.: 13/162,750

(22) Filed: Jun. 17, 2011

Related U.S. Application Data
(60) Provisional application No. 61/356,149, filed on Jun. 18, 2010.

(51) Int. Cl. A61B 1/06; A61B 1/32

(52) U.S. Cl. 600/205; 600/246

(57) ABSTRACT

A veress needle including a housing, a first optical insert for contact viewing, and a second optical insert for distance viewing. The housing including one or more insufflation conduits and an elongate member. The one or more insufflation conduits being adapted to permit the passage and reception of at least one fluid therethrough. The housing defines a passage therethrough. The first and second optical inserts are operably associated with the housing and removably positionable within the passage of the housing while the elongate member remains positioned within tissue. Each optical insert includes an optical assembly having one or more lenses and an ocular. The one or more lenses are adapted to receive an image and transmit the image to the ocular so that the image is perceptible through the ocular.

### CROSS REFERENCE TO RELATED APPLICATION

The present application claims the benefit of and priority to U.S. Provisional Application Ser. No. 61/356,149 filed on Jun. 18, 2010, the entire contents of which are incorporated herein by reference.

### BACKGROUND

1. Technical Field

This application generally relates to the field of laparoscopic surgery. More particularly, the present disclosure relates to veress needles for laparoscopic visualization of a body cavity.

2. Description of Related Art

In laparoscopic procedures, surgery is performed in the interior of the abdomen through a small incision. In endoscopic procedures, surgery is performed in any hollow viscus of the body through a narrow tube or cannula inserted through a small entrance incision in the skin. Since surgical procedures in the abdominal cavity of the body require insufflating gases to raise the cavity wall away from vital organs, the procedure is usually initiated by use of a veress needle through which a gas, such as CO₂, is introduced into the body cavity, thereby creating a pneumoperitoneum. The gas provides a positive pressure which raises the inner body wall away from internal organs, thereby providing the surgeon with a region within which to operate and avoid unnecessary contact with the organs by the instruments inserted into the body cavity.

Moreover, laparoscopic and endoscopic procedures often require the surgeon to visually inspect and magnify organs, tissue, and vessels both near and far removed from the incision. However, in order permit a more comprehensive visual inspection and magnification of the body cavity including material far removed from the incision, the surgeon typically relies on a camera or endoscope that is inserted through a separate incision.

In this manner, veress needles and endoscopes have each provided significant clinical benefits. Nonetheless, improvements are possible, for example, by reducing the complexity of manufacture and/or application.

### SUMMARY

Accordingly, the present disclosure is directed to a veress needle that includes a housing, a first optical insert, and a second optical insert. The housing includes one or more insufflation conduits and an elongate member. The housing may include one or more seals adapted to permit the sealed reception of one or both of the first and second optical inserts. The one or more insufflation conduits are adapted to permit the passage and reception of one or more fluids therethrough. The elongate member extends from the housing and is adapted for insertion within tissue to access an underlying tissue site. The housing defines a passage therethrough. The first and second optical inserts are operably associated with the housing and removably positionable within the passage of the housing while the elongate member remains positioned within tissue.

Each optical insert includes an optical assembly having one or more lenses and an ocular. The optical assembly includes a fiber optic image bundle. The optical assembly defines a chamber between the fiber optic image bundle and the one or more lenses. The chamber may include index matching material. The one or more lenses may be a standard lens. The one or more lenses may be diffractive lenses. The one or more lenses are adapted to receive an image and transmit the image to the ocular so that the image is perceptible through the ocular. The one or more lenses of the first optical insert transmit images that are in direct contact with the distal end of the first optical insert to the ocular of the first optical insert for contact viewing. The one or more lenses of the second optical insert transmit images that are disposed at a distance from the distal end thereof to the ocular of the second optical insert for distance viewing. The first and second optical inserts are interchangeably positionable within the passage of the housing.

The optical assembly may include one or more fiber optic light guides adapted to permit the passage and reception of light therethrough. The optical assembly may include one or more light emitting diodes that transmit light through the one or more light guides. The light may be emitted from an external light source operably associated with the optical assembly. The optical assembly may include a beam splitter operably associated with the ocular.

In one aspect, a method of viewing an underlying tissue site includes providing a veress needle including a housing having an elongate member extending therefrom and first and second optical inserts. The method further includes positioning the elongate member within tissue to access an underlying tissue site. The method involves positioning one of the first and second optical inserts within the housing. The method includes removing the first or second optical insert from the housing. The method further includes selectively positioning the other of the first and second optical inserts within the housing. The method involves viewing the underlying tissue site with one or more of the first and second optical inserts. Typically, the contact viewing insert is used to assess the initial state of the tissue site during insertion of the needle and the distance viewing insert is used for viewing within the pneumoperitonium. The method may include providing illumination through one or more of the first and second optical inserts. The method may involve insufflating the underlying tissue site.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a perspective view of one embodiment of a veress needle in accordance with the present disclosure;

FIG. 2 is an exploded perspective view, with parts separated, of the veress needle of FIG. 1 illustrating a housing and one embodiment of an optical assembly thereof;

FIG. 3 is a side cross-sectional view of the veress needle of FIGS. 1 and 2 with the optical assembly being positioned within the housing;

FIG. 4 is a side cross-sectional view of the optical assembly for contact viewing;

FIG. 5 is a side cross-sectional view of the housing; and

FIG. 6 is a side cross-sectional view of another embodiment of an optical assembly for distance viewing and showing a different embodiment of an illumination assembly.

It should be understood that veress needles are extremely slender instruments and that, for clarity, the drawings above are not to scale. Further, a veress needle typically has a sharpened or beveled distal end to enable penetration of tissue.

### DETAILED DESCRIPTION

Detailed embodiments of the present disclosure are disclosed herein; however, the disclosed embodiments are merely exemplary of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Particular embodiments of the present disclosure will be described herein with reference to the accompanying drawings. As shown in the drawings and as described throughout the following description, and as is traditional when referring to relative positioning on an object, the term “proximal” refers to the end of the apparatus that is closer to the user and the term “distal” refers to the end of the apparatus that is farther from the user. In the following description, well-known functions or constructions are not described in detail to avoid obscuring the present disclosure in unnecessary detail.

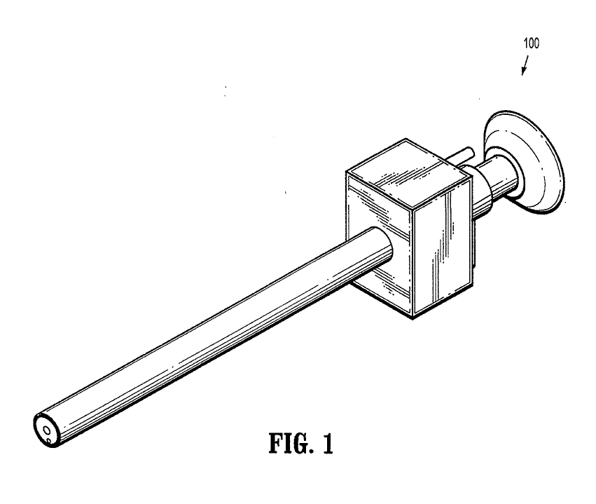

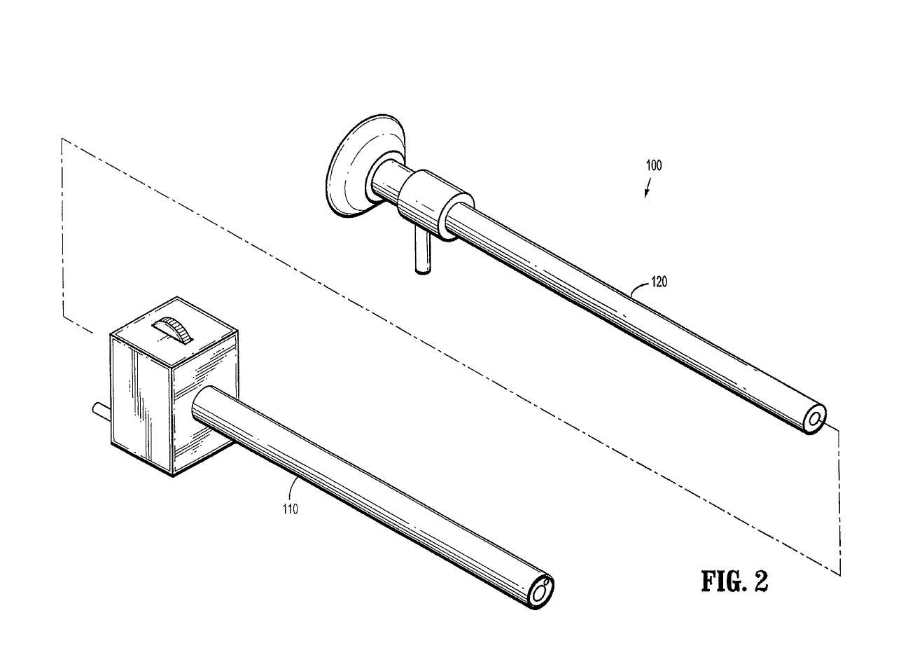

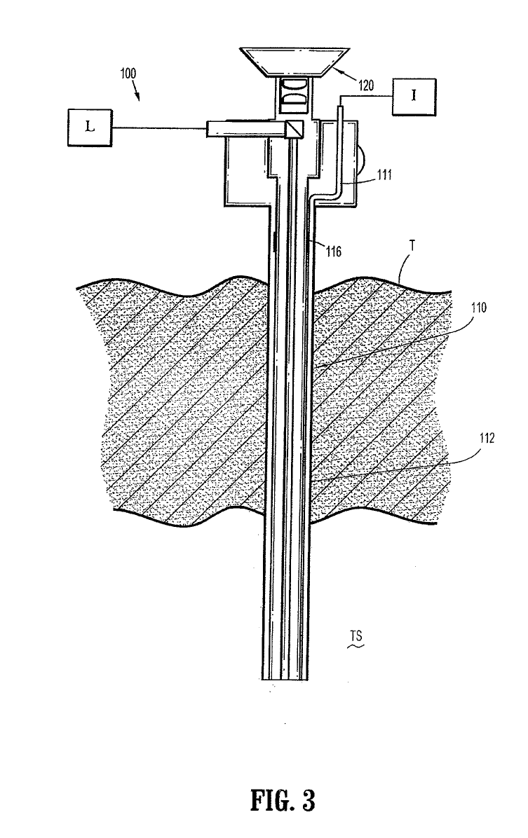

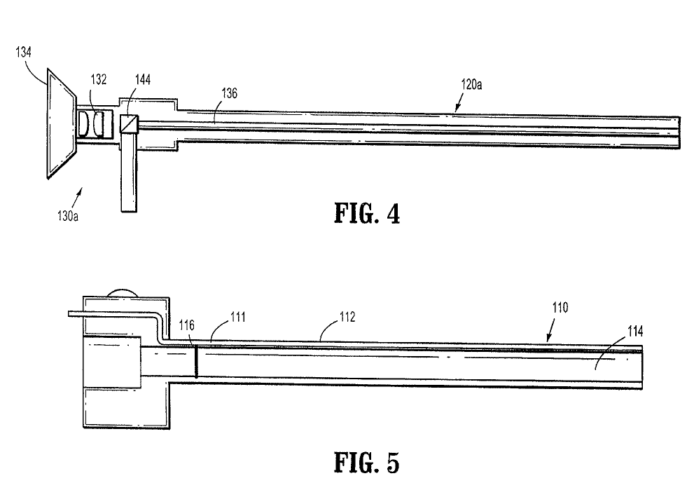

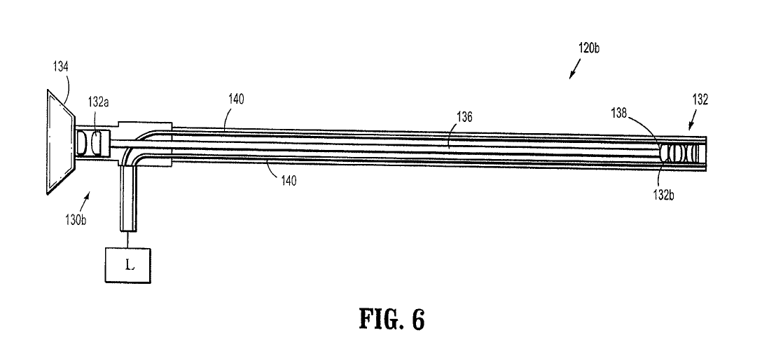

Referring now to the drawings, FIG. 1 illustrates one embodiment of a veress needle which is generally designated as 100. As illustrated in FIG. 2, the veress needle 100 includes a housing 110, and one or more optical inserts 120. In particular, optical inserts 120a (FIG. 4) for contact viewing and 120b (FIG. 6) for distance viewing are alternatively referred to as the one or more optical inserts 120. Referring now to FIGS. 3 and 5, the housing 110 includes one or more insufflation conduits 111 and an elongate member 112. With continued reference to FIG. 3, the one or more insufflation conduits 111 are adapted to permit the passage and reception of one or more fluids, e.g., CO₂, therethrough from an insufflation source “I” in order to create a working space in an underlying tissue site “TS.”

The elongate member 112 extends from the housing 110 and is adapted for insertion within tissue “T” to access the underlying tissue site “TS.” As shown in FIG. 4, the housing 110 defines a passage 114 therethrough. Referring now to FIGS. 2 and 3, each optical insert 120 is operably associated with the housing 110 and is removably positionable within the passage 114 of the housing 110 while the elongate member 112 remains positioned within tissue “T” (FIG. 3). As such, the housing 110 may include one or more seals 116 (FIGS. 3 and 5) that are adapted to permit the sealed reception of each optical insert 120.

Referring again to FIG. 4, one embodiment of an optical insert 120a for contact viewing includes an optical assembly 130a having one or more lenses 132 and an ocular 134. The one or more lenses 132 are adapted to receive images of the underlying tissue site “TS” (FIG. 3) and transmit the images to the ocular 134 so that the images are perceptible through the ocular 134. The optical assembly 130a includes a fiber optic image bundle 136 that transmits images in direct contact with the distal end of the optical insert 120a to the one or more lenses 132. The one or more lenses 132 may be a standard lens, a diffractive lens, a Fresnel lens, or any other suitable lens. The optical assembly 130a may include a beam splitter 144 operably associated with the ocular 134. As shown in FIG. 3, the beam splitter 144 facilitates the transmission of light to the distal end of elongate member 112 when operably coupled to a light source “L,” e.g., a light emitting diode, flashlight, etc. while simultaneously allowing viewing through the ocular 134.

With reference to FIG. 6, another embodiment of an optical insert 120b for distance viewing is substantially similar to optical insert 120a. Optical insert 120b includes an optical assembly 130b having one or more lenses 132 and an ocular 134. In particular, the optical assembly 130b includes one or more proximal and distal lenses 132a, 132b in order to receive and transmit images that are offset a distance from the distal end of the optical insert 120b. In this manner, the optical insert 120b enables a clinician to perceive images within the underlying tissue site “TS” (FIG. 3) that are at a distance, e.g., the optical insert 120b enables distance viewing at an appropriate operative distance. The optical assembly 130b includes a fiber optic image bundle 136 that transmits images received by the one or more distal lenses 132b from the one or more distal lenses 132b to the one or more proximal lenses 132a and the ocular 134. The one or more lenses 132 may be a standard lens, a diffractive lens, a Fresnel lens, or any other suitable lens.

With continued reference to FIG. 6, a chamber 138 may be defined between the fiber optic image bundle 136 and the one or more distal lenses 132b. The chamber 138 may include index matching material. In addition, illumination may also be provided to or within the optical insert 120b via a light source “L” operably associated therewith. The optical assembly 130 may include one or more fiber optic light guides 140 adapted to permit the passage and reception of light therethrough.

In operation, the underlying tissue site “TS” may be accessed by positioning the elongate member 112 within tissue “T.” The underlying tissue site “TS” may then be insufflated to provide a working space within the body cavity. With the veress needle 100 in situ and with the optical insert 120a positioned within the housing 110, images of material, e.g., tissue, organs, vessels, etc., within the underlying tissue site “TS” in contact with or in close proximity to the distal end of the optical insert 120a, may be viewed. Alternatively, the optical insert 120b may be positioned within the housing 110 so that images of material at a distance relative to the distal end of the optical insert 120b may be viewed. Whichever optical insert 120 is positioned within the housing 110 may then be removed therefrom after viewing the underlying tissue site “TS” at the respective distal or proximal proximity. The same or a different optical insert 120 may then be selectively positioned within the housing 110. Each of these optical inserts 120a, 120b are interchangeably positionable within the passage 114 of the housing 110. In this respect, the one or more seals 116 permit the sealed reception and passage of each optical insert 120 through the housing 110 when the optical inserts 120 are interchanged. Each optical insert 120 may have one or more lenses 132 that have the same or different powers and thicknesses. Optical insert 120a enables a clinician to perceive different and/or more comprehensive details of images within the tissue “T.” Optical insert 120b enables a clinician to perceive different and/or more comprehensive details of images within an underlying tissue site “TS.”

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of the presently disclosed embodiments. Thus the scope of the embodiments should be determined by the appended claims and their legal equivalents, rather than by the examples given.

What is claimed is:

1. A veress needle, comprising:
a housing including at least one insufflation conduit and an elongate member, the at least one insufflation conduit being adapted to permit the passage and reception of at least one fluid therethrough, the elongate member extending from the housing and being adapted for insertion within tissue to access an underlying tissue site, the housing defining a passage therethrough; and
first and second optical inserts operably associated with the housing and removably positionable within the passage while the elongate member remains positioned within tissue, each optical insert including an optical assembly having at least one lens and an ocular, the at least one lens adapted to receive an image and transmit the image to the ocular so that the image is perceptible through the ocular.

2. The veress needle according to claim 1, wherein the at least one lens of the first optical insert transmits images that are in direct contact with the distal end of the first optical insert to the ocular of the first optical insert and the at least one lens of the second optical insert transmits images that are disposed at a distance from the distal end thereof to the ocular of the second optical insert.

3. The veress needle according to claim 1, wherein the first and second optical inserts are interchangeably positionable within the passage of the housing.

4. The veress needle according to claim 1, wherein the optical assembly includes at least one fiber optic light guide adapted to permit the passage and reception of light therethrough.

5. The veress needle according to claim 4, wherein the optical assembly includes at least one light emitting diode that transmits light through the at least one light guide.

6. The veress needle according to claim 4, wherein the light is emitted from an external light source operably associated with the optical assembly.

7. The veress needle according to claim 1, wherein the optical assembly includes a beam splitter operably associated with the ocular.

8. The veress needle according to claim 1, wherein the optical assembly includes a fiber optic image bundle.

9. The veress needle according to claim 8, wherein the optical assembly defines a chamber between the fiber optic image bundle and the at least one lens.

10. The veress needle according to claim 9, wherein the chamber includes index matching material.

11. The veress needle according to claim 1, wherein the at least one lens is a diffractive lens.

12. The veress needle according to claim 1, wherein the at least one lens is a standard lens.

13. The veress needle according to claim 1, wherein the housing further includes at least one seal adapted to permit the sealed reception of at least one of the first and second optical inserts.

14. A method of viewing an underlying tissue site, comprising the steps of:
providing a veress needle including a housing having an elongate member extending therefrom and first and second optical inserts;
positioning the elongate member within tissue to access an underlying tissue site;
positioning one of the first and second optical inserts within the housing;
removing the first or second optical insert from the housing;
selectively positioning the other of the first and second optical inserts within the housing; and
viewing the underlying tissue site with at least one of the first and second optical inserts.

15. The method of claim 14, further comprising the step of:
providing illumination through at least one of the first and second optical inserts.

16. The method of claim 14, further comprising the step of:
insufflating the underlying tissue site.

17. The method of claim 14, wherein:
the first optical insert is adapted for contact viewing and the second optical insert is adapted for non-contact viewing.

18. The method of claim 17, wherein:
the second optical insert is adapted for distance viewing.
